(19) United States
(12) Patent Application Publication
(10) Pub. No.: US 2009/0270904 A1
(43) Pub. Date: Oct. 29, 2009

(54) Remotely Adjustable Gastric Banding System

(76) Inventors: Janel A. Birk, Oxnard, CA (US); Sean Snow, Capinteria, CA (US)

Correspondence Address: ALLERGAN, INC., 2525 DUPONT DRIVE, T2-7H, IRVINE, CA, 92612-1599, US

(21) Appl. No.: 12/428,311

(22) Filed: Apr. 22, 2009

Related U.S. Application Data
(60) Provisional application No. 61/047,356, filed on Apr. 23, 2008.

(51) Int. Cl. A61M 29/02

(52) U.S. Cl. 606/192

(57) ABSTRACT

Presently described are systems for facilitating obesity control comprising: a gastric banding device including at least one inflatable member for containing fluid; a fluid reservoir; an implantable pump unit in communication with the fluid reservoir for controlling pressure within the inflatable member, the implantable pump unit including a piezoelectric diaphragm pump and a remote control device capable of communicating with the pump.

### CROSS REFERENCE TO RELATED APPLICATIONS

This application claims the benefit of U.S. provisional patent application No. 61/047,356, filed Apr. 23, 2008, the entire disclosure of which is incorporated herein by reference.

### FIELD OF THE INVENTION

The present description generally relates to medical systems and apparatus and uses thereof for treating obesity and/or obesity-related diseases, and more specifically, relates to remotely adjustable gastric banding systems.

### BACKGROUND

Adjustable gastric banding apparatus have provided an effective and substantially less invasive alternative to gastric bypass surgery and other conventional surgical weight loss procedures. Despite the positive outcomes of invasive weight loss procedures, such as gastric bypass surgery, it has been recognized that sustained weight loss can be achieved through a laparoscopically-placed gastric band, for example, the LAP-BAND® (Allergan, Inc., Irvine, Calif.) gastric band or the LAP-BAND AP® (Allergan, Inc., Irvine, Calif.) gastric band. Generally, gastric bands are placed about the cardia, or upper portion, of a patient's stomach forming a stoma that restricts food's passage into a lower portion of the stomach. When the stoma is of an appropriate size that is restricted by a gastric band, food held in the upper portion of the stomach provides a feeling of satiety or fullness that discourages overeating. Unlike gastric bypass procedures, gastric band apparatus are reversible and require no permanent modification to the gastrointestinal tract.

Over time, a stoma created by a gastric band may need adjustment in order to maintain an appropriate size, which is neither too restrictive nor too passive. Accordingly, prior art gastric band systems provide a subcutaneous fluid access port connected to an expandable or inflatable portion of the gastric band. By adding fluid to or removing fluid from the inflatable portion by means of a hypodermic needle inserted into the access port, the effective size of the gastric band can be adjusted to provide a tighter or looser constriction.

Naturally, it would be desirable to allow for non-invasive adjustment of gastric band constriction, for example, without the use of a hypodermic needle. Thus, remotely adjustable gastric banding systems have been proposed and are described herein.

### SUMMARY

Generally described herein are remotely adjustable and powered gastric band systems, and methods of use thereof. The apparatus, systems and methods described herein aid in facilitating obesity control and/or treating obesity-related diseases while being non-invasive once implanted.

The systems generally comprise a gastric band having at least one inflatable member or bladder and a high precision pump unit in communication with or couplable to at least one inflatable member of the gastric band.

The high precision pump unit described herein is an implantable, high precision pump device capable of adjusting, and even fine tuning, the stoma created by the gastric band. In some embodiments, high precision pump unit may be substantially passive and requires minimal power to operate. Further, the high precision pump unit is configured and structured to enable tightening and/or loosening of a gastric band by moving a metered amount of fluid between an implantable reservoir and the at least one inflatable member of the gastric band. The high precision pump unit can also be controlled by a handheld remote control unit operated by a clinician, patient or caretaker. The high precision pump unit can further include an incorporated override port for enabling conventional adjustment of the band using a hypodermic needle inserted therein, if necessary.

In one example embodiment, the high precision pump unit comprises at least one high precision piezoelectric, unidirectional pump. The pump is unidirectional in that it allows flow only in a direction from the reservoir to the at least one inflatable member or bladder of the gastric band. The pump is effective in pumping small, metered volumes of fluid into the at least one inflatable member. At least one check valve may be provided within the high precision pump unit for preventing flow in an opposing direction of the desired flow.

Additionally, high precision pump unit further includes a parallel flow line for allowing fluid flow in a direction from the at least one inflatable member to the reservoir, thus decreasing the constriction of the at least one inflatable member against the stoma.

In one example embodiment described herein is a system for facilitating obesity control comprising: a gastric banding device including at least one inflatable member for containing fluid; a fluid reservoir; an implantable pump unit in communication with the fluid reservoir for controlling pressure within the at least one inflatable member, the implantable pump unit including a metering assembly and a receiver assembly in communication with the metering assembly; and a remote control device capable of communicating with the receiver assembly.

In another example embodiment, the metering assembly comprises a piezoelectric pump, or alternatively a piezoelectric diaphragm pump or unidirectional pump. The pump, in some embodiments comprises a flexible membrane. In yet another example embodiment, the piezoelectric pump is effective in drawing fluid away from the reservoir and toward the at least one inflatable member. Alternatively, the piezoelectric pump further comprises a valve effective to pass fluid in a direction from the at least one inflatable member to the reservoir.

In a further example embodiment, the system further comprises a pressure sensor effective in sensing a pressure of the at least one inflatable member or an access port for enabling manual adjustment of a volume or a pressure of fluid in the at least one inflatable member.

In still further example embodiments, the access port is an integral part of the pump unit and implantable pump unit does not require a battery to operate. Further still, in other example embodiments, the system comprises substantially no implanted ferromagnetic materials and is MRI safe.

In yet a further example embodiment, the pump unit comprises a first fluid line including a one-way pump for passing fluid in a first direction and a second fluid line in parallel with the first line including a valve for passing fluid in an opposing direction.

In one example embodiment of the invention, an implantable high precision pump unit for use with a gastric band is provided. The pump unit generally comprises a inductively powered, piezoelectric pump, a first valve in line with the pump, a second valve in parallel with the pump; electronics in communication with the pump and first and second valves; an override port; and a housing containing the pump, first and second valves, electronics and override port, wherein the pump unit is operable without the use of an implanted energy storage device.

In another embodiment, the high precision pump unit is controlled by an external remote control unit. The high precision pump unit can be inductively powdered by an external remote control unit in one example embodiment.

In still another example embodiment, the housing of the high precision pump unit is hermetically sealed. In yet another example embodiment, the override port is manufactured at a position that is located away from at least one tube emanating from the high precision pump unit. Even further still, in another example embodiment, the at least one pump can deliver fluid to at least one gastric band from at least one reservoir, both connected to the high precision pump unit via at least one tube.

In one example embodiment described herein is a system for facilitating obesity control comprising: an implantable gastric banding device including at least one inflatable member for containing fluid and restricting a patient's cardia; an implantable fluid reservoir; an implantable pump unit in communication with the fluid reservoir and the gastric band device for controlling pressure within the at least one inflatable member, the implantable pump unit including at least one piezoelectric pump and at least one valve in a parallel configuration and a receiver in direct communication with at least one piezoelectric pump and at least one valve; and an external remote control device capable of communicating with the receiver assembly and powering the implantable pump unit. In another example embodiment, the system further comprises an override port located on the implantable pump unit.

### BRIEF DESCRIPTION OF THE DRAWINGS

FIG. 1 illustrates an overall schematic view of an example configuration of components according to the present description.

FIG. 2 illustrates en example configuration of the internals of a high precision pump unit.

FIGS. 3A and 3B illustrate cross-sections of an example pump.

FIGS. 4A and 4B illustrate cross-sections of an example valve.

FIGS. 5A and 5B illustrate cross-sections of an example pressure sensor.

FIG. 6 illustrates the filling of a gastric band using the systems described herein.

FIG. 7 illustrates the draining of a gastric band using the systems described herein.

### DETAILED DESCRIPTION

The present invention generally provides remotely adjustable gastric banding systems, for example, for treatment of obesity and obesity related conditions, as well as systems for controlling inflation of gastric banding systems.

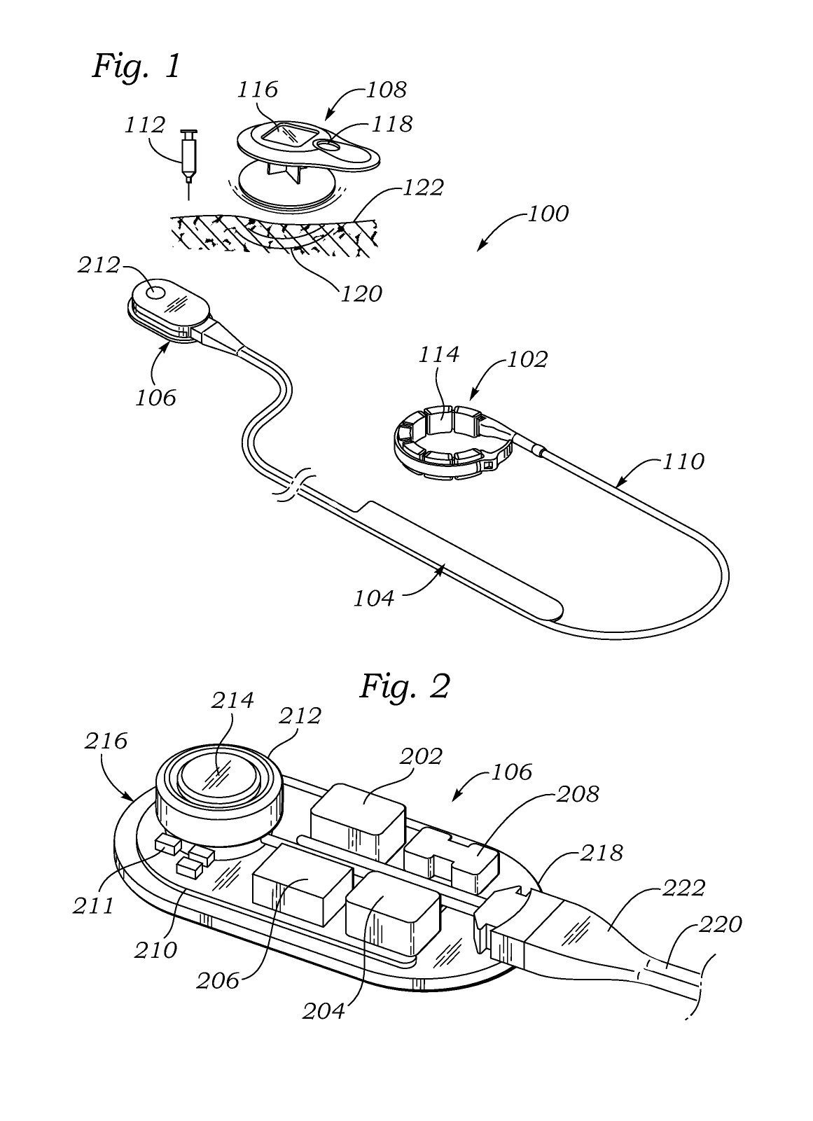

Turning now to FIG. 1, a gastric banding system 100 in accordance with one embodiment of the invention generally includes a gastric band 102, reservoir 104, high precision pump unit 106, remote controller unit 108 and tubing 110. Each of the components of system 100, other than remote controller unit 108, is implantable in a patient using conventional surgical techniques. High precision pump unit 106 can be used to replace or complement a conventional access port for adjusting inflation of gastric band 102. In some embodiments, the system includes an override port 112 which can be used, for example, with a hypodermic needle 112, to fill and drain the gastric band 102.

High precision pump unit 106 is connected to reservoir 104 and gastric band 102 via tubing 110, and can move precisely metered volumes of saline in or out of gastric band 102. Moving saline into gastric band 102 causes inflation of at least one bladder, or inflatable member 114 and constricts around the cardia, or upper portion of the stomach, forming a stoma that restricts the passage of food into a lower portion of the stomach. This stoma can provide a patient with a sensation of satiety or fullness that discourages overeating. In contrast, moving saline out of at least one inflatable member 114 of gastric band 102 contracts the pressure around the cardia and allows a stoma to be at least partially released and regains the patient's hunger sensation.

High precision pump unit 106 is implanted within a patient, and therefore, is non-biodegradable. The encasement of the unit is hermetically sealed from the in situ environment and formed at least partially of any rugged plastic material including, polypropylene, cyclicolephin co-polymer, nylon, and other compatible polymers and the like or at least partially formed of a non-radioopaque metal such as titanium. The encasement has a smooth exterior shape, with no jagged edges, to minimize foreign body response and tissue irritation. The unit itself is also sterilizable, preferably dry heat sterilizable before implantation.

The internal components of high precision pump unit 106 are illustrated in FIG. 2. The encasement of high precision pump unit 106 has an internal volume of less than about 0.75 in³, preferably less than about 0.5 in³ (8 cm³). Exemplary internal features of high precision pump unit 106 that fit within the encasement include first valve 202, second valve 204, pump 206, pressure/flow sensor 208, electronics board 210 including an antenna 211, and override port 212. The internal components of high precision pump unit 106 can be arranged in any fashion appropriate for delivering and removing precise amounts of fluid from gastric band 102 and reservoir 104.

Pump 206 can be actively or passively driven. If pump 206 is actively driven, a local power source such as a battery (not illustrated) is provided to drive pump 206. If pump 206 is passively driven, it may be inductively powered by a device external to high precision pump unit 106. In an exemplary configuration, pump 206 is passively driven through inductive power from remote controller unit 108.

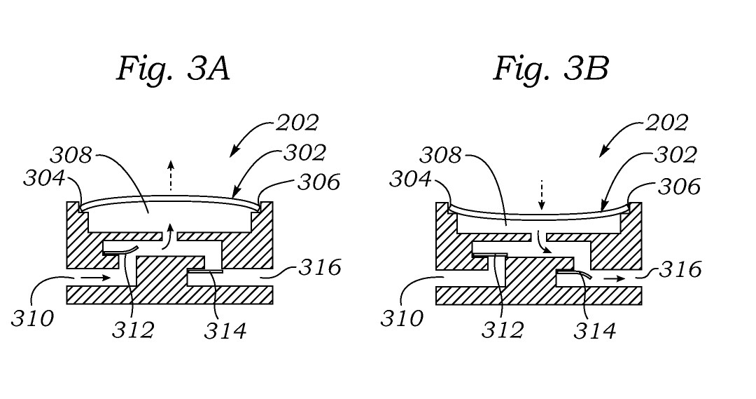

In one example embodiment, pump 206 is an inductively powered, electrically driven, positive displacement piezoelectric pump. Pump 206 provides the means to move fluid into gastric band 102. With reference to FIG. 3A, when a pumping sequence is initiated to move fluid into at least one inflatable member 114 of gastric band 102, piezoelectric bending membrane 302 is charged and is deflected upwards. Since piezoelectric bending membrane 302 is sealed along first edge 304 and second edge 306, a negative pressure is created which pulls fluid into chamber 308 from inlet line 310. First check valve 312 opens to permit flow while second check valve 314 remains closed.

Then, referring to FIG. 3B, when electric potential is reversed to pump 202, piezoelectric bending membrane 302 deflects downward and forces fluid out of chamber 308. As fluid is forced out of chamber 308, first check valve 312 is forced closed while second check valve 314 opens forcing fluid out of exit line 316. In a preferred embodiment, inlet line 310 is connected either directly or indirectly to reservoir 104 and exit line 316 is connected either directly or indirectly to gastric band 102.

Pump 202 can move fluid from the reservoir 104 to gastric band 102 at rates higher than about 0.5 cc/min, for example, higher than about 1 cc/min for band pressures less than about 20 psi (about 138 kPa) relative to the reservoir pressure. Alternatively, fluid can be drained from gastric band 102 to reservoir 104 at rates higher than about 0.5 cc/min, for example, higher than about 1 cc/min for band pressures above about 0.2 psi (about 1.38 kPa).

Reservoir 104 is a soft, collapsible balloon made of a biocompatible polymer material, for example, silicone, which holds a reserve of a biocompatible fluid, for example, saline, to allow for adjustments in the size of gastric band 102. The reservoir is preferably fully collapsible and can contain the extra fluid required to increase the volume of gastric band 102 to therapeutic levels. Further, reservoir 104 also may have excess capacity so gastric band 102 may be fully drained into it without reservoir 104 being filled beyond its maximum capacity.

The fluids used within the systems of the present description include any fluid that is biocompatible. The fluid has no adverse effect on the patient in the unlikely event that a leak emanates from the system. The fluid can simply be water or any biocompatible polymer oil such as caster oil. In an example embodiment, the fluid is saline.

Tubing 110 is any biocompatible flexible tubing that does not degrade in vivo. Tubing 110 is configured to withstand hydraulic forces up to about 30 psi (about 206 kPa) without leakage. This hydraulic pressure tolerance is true of the entire fluid path of the systems described herein. Although the systems described herein do not generally leak, if they do, fluid is not lost at a rate greater than about 0.2 cc/yr, or about 0.1 cc/yr.

Other biocompatible and biostable polymers which are useful for forming reservoir 104 and tubing 110 include polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

First valve 202 and second valve 204, illustrated in FIG. 2, can be any valve known in the art to allow precise delivery of fluid and precise flow rates therethrough. Preferably, first valve 202 and second valve 204 only allow fluid to move in one direction, therefore, the two valves are situated in parallel with high precision pump unit 106 allowing fluid to drain back from gastric band 102. Further, first valve 202 and second valve 204 should have a precision orifice that restricts the flow rate to a well-characterized, precise amount.

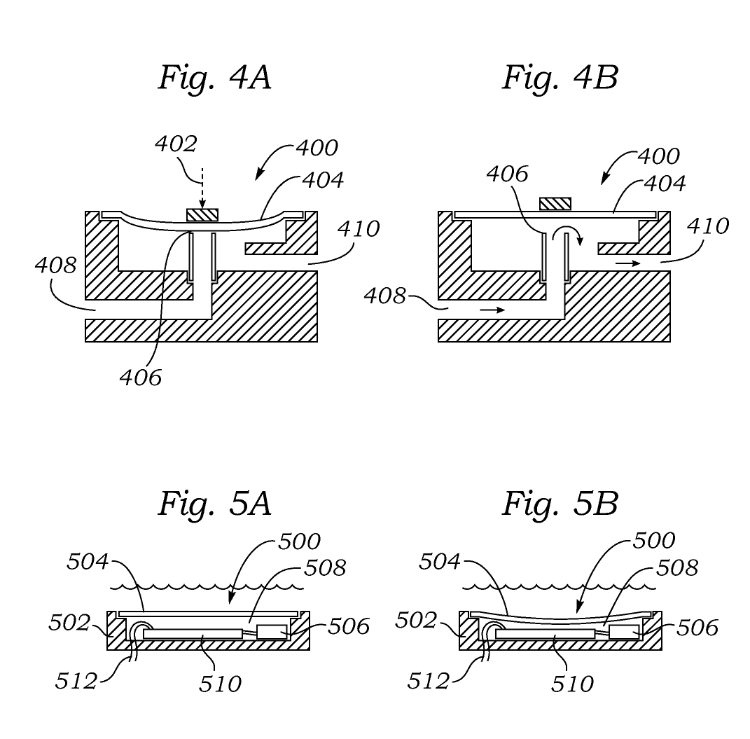

FIGS. 4A and 4B illustrate an exemplary valve 400 according to an embodiment of the invention. Valve 400 may be used in place of one or both of first valve 202 and second valve 204.

Referring to FIG. 4A, valve 400 is biased in a closed position, for example, by spring preload force 402 acting on a seal 404, for example, a flexible silicone seal 404. For example, spring preload force 402 pushes flexible silicone seal 404 into sealing engagement with a valve seat 406. When valve 400 is sealed as shown in FIG. 4A, fluid cannot pass from valve inlet 408 to valve exit 410. Now referring to FIG. 4B, when flow is desired, a signal is sent to valve actuator (not shown) which removes spring preload force 402 and permits flexible silicone seal 404 to relax into an open position, out of sealing engagement with valve seat 406. Fluid is then free to flow from valve inlet 408 to valve exit 410 until valve 400 is closed, for example, by reapplication of spring preload force 402.

Turning back now to FIGS. 2, 6 and 7, the system 100 may further comprise at least one flow or pressure sensor 208 disposed, for example, within high precision pump unit 106. In an exemplary embodiment, two pressure sensors are situated within the fluid pathway between first valve 202 and second valve 204 and gastric band 102. During a no-flow condition, both of the pressure sensors may be used to measure pressure thereby providing the benefits of redundancy and averaging.

For example, sensing or measuring the pressure within the fluid pathway of system 100 provides diagnostic uses. Clinicians can measure pressure while a patient drinks water, recording and analyzing resulting pressure fluctuations which can help determine if gastric band 102 is too restrictive. A band that is too restrictive can also be confirmed by the patient's response (generally discomfort) upon drinking the water, and can then be appropriately adjusted. Further, sensing or measuring pressure in the system 100 can be useful in diagnosing system leaks or obstructions. For example, if the pressure consistently drops over an extended period of time, the clinician can diagnose a leak within the system and plan for an appropriate treatment to fix the problem. In contrast, if there is an obstruction within the system with a sustained pressure rise over time, the clinician can diagnose an obstruction within the system and plan for an appropriate treatment to fix the problem.

FIGS. 5A and 5B illustrate an exemplary pressure sensor 500 according to the present invention which can be used in place of pressure sensor 208 in the system 100. Referring to FIG. 5A, pressure sensor 500 is hermetically sealed and comprises housing 502, for example, a metallic housing, and membrane 504, both of which may comprise titanium or other non-radioopaque material. Membrane 504 flexes in response to a signal, which affects pressure transducer 506. Space inside housing 502 may contain degassed silicone oil 508 which serves as an incompressible pressure transfer medium. As pressure outside pressure sensor 500 is increased, as illustrated in FIG. 5B, membrane 504 deflects downward increasing the pressure of the degassed silicone oil 508. Pressure transducer 506 converts pressure changes into changes in capacitance which are then detected by electronics board 510. Electronics board 510 converts the sensitive analog pressure signal into a hardy digital signal that can pass through housing 502 via signal wire 512 which is substantially immune to typical levels of electrical noise.

Override port 212, illustrated in FIG. 1 and FIG. 2 is an optional feature of some embodiments of the present invention. Override port 212 can be manufactured from any metal, preferably titanium or another non-radioopaque and is accessible by insertion of non-coring, hypodermic needle 112 (FIG. 1) through a self-sealing septum 214. Override port 212 allows a clinician to use hypodermic needle 112 or a standard syringe to fill or drain gastric band 102. Further, override port 212 may be located on distal end 216 of high precision pump unit 106, for example, at a position substantially opposite from proximal end 218 where tubing 220 extends from high precision pump unit 106. This placement of override port 212 thereby reduces possible occurrences of a needle damaging tubing 220. Extension body 222 emanating from high precision pump unit 106 further protects tubing 220 from accidental needle sticks.

The systems and apparatus described herein further include remote controller unit 108, which provides access to system data and functions and is an external, handheld, reusable battery-powered device. Remote controller unit 108 can be made of any rugged plastic material including, polypropylene, cyclicolephin co-polymer, nylon, and other compatible polymers and the like. The controller unit is not implanted within the patient so hermetic sealing of the unit is not required. However, remote controller unit 108 is preferably at least water resistant, if not waterproof, and can be cleaned using standard hospital disinfectants without damage to the unit.

Further, remote controller unit 108 has a user interface including at least one display 116 and at least one user input 118. In some example embodiments, display 116 and user input 118 are combined in the form of a touch screen with a color display. In other embodiments, the display is grayscale. Remote controller unit 108 permits a clinician or a patient to navigate through menu driven screens used for data entry, data collection, and high precision pump unit 106 control.

Remote controller unit 108 is capable of communicating with high precision pump unit 106. “Capable of communicating” as used herein refers to the remote controller's ability to establish communications with high precision pump unit 106 yet still have the ability to break communication and the systems described herein still function. To establish communication, in one example embodiment, once remote controller unit 108 is initialized, display 116 shows a searching query for a nearby high precision pump unit 106. As remote controller unit 108 is brought into range of high precision pump unit 106, a symbol displays the strength of the communication link. Once stable communications have been acquired, display 116 shows the serial number of the system so a clinician can verify they have the appropriate patient records in hand. If the patient requires a tightening of gastric band 102, the clinician can enter the amount of the desired volume increase. Remote controller unit 108 can also display the current volume within gastric band 102 and indicate the new volume as gastric band 102 fills. Display 116 can also indicate desired and actual volumes during gastric band 102 draining.

To verify the appropriate adjustment has been made to the system, the clinician can set remote controller unit 108 into pressure monitor mode and request that the patient drink water. Display 118 shows a real time graph of the pressure measured within gastric band 102. This diagnostic tool may show higher pressures and warning messages if gastric band 102 has been over-tightened.

Remote controller unit 108 can synchronize and charge when coupled with a charging cradle or docking station. This docking station provides the ability to recharge remote controller unit's 108 rechargeable battery and provides a link to download information to a personal computer such as the adjustment history of a patient. Other data that can be stored on remote controller unit 108 and downloaded from high precision pump unit 106 includes, but is not limited to serial number, gastric band size, patient information, firmware version and patient adjustment history. This data can be downloaded directly to a patient tracking database for ease tracking.

Any data stored on remote controller unit 108 or within high precision pump unit 106 can be electronically secured. In other words, security measures can be put in place to keep the data confidential, including communication between high precision pump unit 106 and remote controller unit 108. Security measures can include computer generated algorithms that prevent intrusion by outside parties.

High precision pump unit 106 can contain a micro-fluidic pump with active valves. In such an embodiment, high precision pump unit 106 is a passive device that can only be powered by remote controller unit 108 when it is in close proximity. For example, in one example embodiment, remote controller unit 108 may be configured to power and communicate with high precision pump unit 106 at any distance less than about 8 inches, preferably less than about 4 inches (about 10.2 cm) of tissue plus about 4 inches, preferably about 2 inches (about 5.1 cm) of air. Power and communications can be tailored to transmit over longer distances or can be tailored to have remote controller unit 108 placed on the skin adjacent to the high precision pump unit 106.

Further, remote controller unit 108 can inductively power and telemetrically control high precision pump unit 106. Remote controller unit 108 may be configured to provide continuous power to high precision pump unit 106. A dedicated microcontroller within remote controller unit 108 monitors the amount of power that is transmitted. Further still, a power management system may be implemented to optimize energy transmission between remote controller unit 108 and high precision pump unit 106 relative to their separation distance. For example, the power transmission may automatically decrease as remote controller unit 108 is closer to high precision pump unit 106, and may be increased as the distance is increased. This minimizes wasted energy, and energy exposure to the patient.

High precision pump unit 106 is a passive device which may be entirely controlled and powered by remote controller unit 108. Antenna 211 on electronics board 210 housed within high precision pump unit 106 and remote controller unit 108 are coupled to allow the transmission of power 122 through skin 124 (as illustrated in FIG. 1). The power issued from remote controller unit 108 is continually monitored by a dedicated microprocessor to ensure that power transmission is minimized to the lowest level required for operation. To minimize the power transmission and to optimize command communication, high precision pump unit 106 and remote controller unit 108 have a channel frequency dedicated to command communication and a separate channel frequency dedicated to power transmission. The command communication can be configured, for example, to take place at about 402-406 MHz while the power transmission, for example, takes place at about 400 kHz. This command communication adheres to the frequency and power standards set by the Medical Implant Communications Service. To ensure accuracy, communication and control commands are verified by error check algorithms prior to data reporting or command implementation.

A portion of electronics board 210 within high precision pump unit 106 is devoted to conditioning and managing the power received at antenna 211 or from a local battery. Communication electronics manage the bidirectional transmissions with timing verification and error checking. Controller circuits of electronics board 210 send commands to first valve 202, second valve 204, pump 206, and pressure/flow sensor 208 and receive data back from pressure/flow sensor 208. Electronics board 210 can be encased in a biocompatible sealant if further protection, or redundant protection, is necessary.

The systems and apparatus described herein use common surgical techniques to place the components in their respective positions within a patient. The surgical techniques may be identical or similar to those used in the placement of conventional gastric banding systems. For example, gastric band 102 may be placed around the stomach using laparoscopic techniques, as known to those of skill in the art. Like a conventional access port, high precision pump unit 106 may be sutured onto the rectus muscle sheath or any other conveniently accessible muscle. In order to achieve a secure attachment of high precision pump unit 106, the unit shall be sutured to the rectus muscle and remain securely attached for forces below about 6 lbf, preferably below about 3 lbf (13.3 N). Tubing 110 from high precision pump unit 106 passes through the rectus muscle into the peritoneal cavity in the same manner as the tubing of a conventional access port.

The systems and apparatus of the present description further allows for a remotely controlled adjustment without needles, non-invasively, by using remote controller unit 108. Also, should remote controller unit 108 be unavailable, damaged, out of power, or in the even of an emergency, an adjustment of gastric band 102 can be performed invasively using a needle. By using override port 212, a clinician can choose to use a standard needle for adjustments. If any of the electronics associated with the systems and apparatus described herein become inoperable, override port 212 can be used to add or remove fluid from gastric band 102. Override port 212 and a syringe or needle can always be used to adjust gastric band 102.

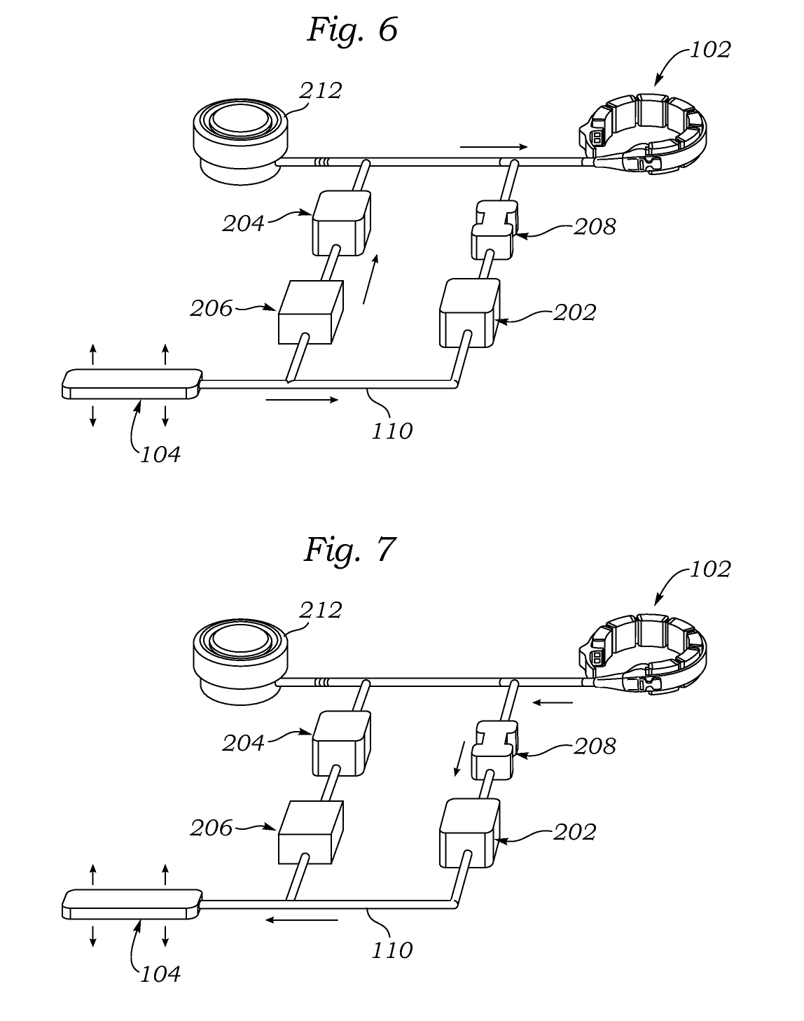

The systems described herein generally function as follows. When a clinician uses remote controller unit 108 to adjust gastric band 102, high precision pump unit 106 initiates a sequence of events to move a precise amount of fluid in the desired direction. Referring to FIG. 6, the system diagram illustrates the filling of gastric band 102. Just before pumping is initiated, second valve 204 in line with pump 206 is opened. Pump 206 is activated pumping fluid into gastric band 102 which creates a differential pressure to draw fluid out of reservoir 104. First valve 202 and pressure/flow sensor 208 are not engaged. Reservoir 104 is collapsible and does not impede the outward flow of fluid. Further, reservoir 104 is sized such that when filled to the maximum recommended fill volume, there is a slight vacuum therein. Once the proper amount of fluid has been transferred from reservoir 104 to gastric band 102, electronics board 210 shuts off pump 206 and closes second valve 204. Gastric band 102 now assumes the new higher pressure.

Alternatively, if the clinician decides to loosen gastric band 102, fluid is released from gastric band 102 and returned to reservoir 104. This process is illustrated in FIG. 7. Once high precision pump unit 106 receives a drain command from remote controller unit 108, first valve 202 behind pressure/flow sensor 208 opens. The amount of fluid released from gastric band 102 is controlled by the amount of time pressure/flow sensor 208 remains open. This timing is based on information from pressure/flow sensor 208 indicating the rate of fluid flow. Once the correct volume of fluid has been transferred, first valve 202 is closed. With both first valve 202 and second valve 204 closed, the volume in gastric band 102 is maintained and the pressure in gastric band 102 can be measured accurately.

When compared to conventional gastric banding systems having standard access ports which exclusively require syringe access, the presently described systems and apparatus offer several benefits. First, for conventional access ports located under a thick layer of fatty tissue, which is generally the case as the devices are generally used to treat obesity, the access port can be difficult to locate. The present systems reduce or eliminate the need for port location as the use of remote controller unit 108 removes the need for adjustment using a syringe.

Secondly, accessing the access port in conventional systems, when there is ambiguity on its location, can cause damage by accidentally puncturing the tubing which connects the access port to the gastric band. This can require a revision surgery in order to repair the punctured tubing. Further, when a conventional access port cannot be located by palpation, x-ray imaging may be required to guide a needle into the access port. Such imaging practices put a patient at risk for x-ray radiation exposure. The present systems and apparatus remove the need for these unnecessary procedures and save the patient from x-ray radiation exposure. As described infra, the present systems and apparatus are compatible with magnetic resonance imaging (MRI), which is much safer for a patient.

In the unlikely event that override port 212 of the present description is used, it may be located away from the tubing connection to gastric band 102 to reduce the potential for tubing needle sticks. High precision pump unit 106 has geometry and a rigid case that can be structured to facilitate the user in locating override port 212 when needed.

In one example embodiment, the systems and apparatus described herein are configured and structured to be compatible with MRI, or MRI safe, at, for example 1.5 T. In the exemplary embodiment shown, the pump unit is entirely inductively powered. The systems utilize no permanent magnets, no long metallic wires or leads, and a minimal or negligible amount of ferrous or ferromagnetic material. The systems are substantially free or contain substantially no ferromagnetic materials. Substantially no ferromagnetic materials refers to materials containing less than about 5%, preferably less than about 1% or 0.1% (w/w) of ferromagnetic material. The resulting systems are thus MRI safe given standard specifications regulating translational and rotational attraction, MRI heating, and imaging artifacts. All materials selected for the systems are preferably selected to be compatible and safe in an MRI environment.

Further, the inductive powering of high precision pump unit 106 requires that energy be passed through body tissue. Since the tissue absorbs a small amount of the energy passing through it, the heating of the tissue can be proportional to the total energy transferred. To ensure that the systems meet standards to minimize tissue heating (below 2° C. above body temperature per ISO 45502), the systems described herein have been designed to use very little power to move the fluid within the system and do not cause excessive heating of the patient's tissue.

The systems may also include a pressure sensor to monitor pressure inside gastric band 102 as needed. Using remote controller unit 108 to communicate with high precision pump unit 106, a clinician can monitor pressure inside gastric band 102, for example, in “real time” during an adjustment of the constriction within gastric band 102. This will allow the clinician to observe the response of gastric band 102 to a patient's adjustment. This may permit a new modality for gastric band adjustment management to monitor pressure as well as volumes during adjustments. With these new pressure sensing capabilities, the clinician can make a determination of whether there is a leak within the system (e.g. zero pressure reading) or whether there is an obstruction in the system (e.g. prolonged pressure rise).

In an example embodiment, high precision pump unit 106 includes a first fluid line including a first pump for passing fluid in a first direction and a second fluid line in parallel with the first fluid line including a first valve and a second pump for passing fluid in an opposing direction. In another example embodiment, the second pump is not needed because pressure from the gastric band allows enough pressure the move the fluid to the reservoir. This use of parallel line configuration allows for filling and draining of gastric band with a minimal number of components and minimal complexity.

The systems and apparatus described herein can achieve at least one of the following features. The total time required to complete a fill or drain of gastric band 102 does not exceed about 10 minutes, more preferably about 5 minutes. The systems are able to adjust the volume in gastric band 102 accurately to within about 0.1 cc or about 10%, whichever is greater. Pressure/flow sensor 208 has a resolution between about 0.010 psi to about 0.025 psi, preferably about 0.019 psi (about 130 Pa).

In one example embodiment of the present description, components of the systems can be replaced without replacing the entire system and subjecting patients to overly invasive surgeries to replace entire systems when a single component is defective or damaged. For example, if high precision pump unit 106 becomes damaged, it can be replaced independently of other components. Alternatively, if gastric band 102 becomes damaged, it can be replaced independently of other components. The same is true of tubing 110 and reservoir 104. Although components can be disconnected for single part replacement, components shall not become dislodged from the tubing for tubing pull-off forces less than about 10 lbf, more preferably, less than about 5 lbf (22.2 N).

The systems described herein meet at least one safety specifications. For example, in the event of any failure of the systems, either no change in gastric band 102 tightness or a loosening of gastric band 102 results. Further, high precision pump unit 106 is biocompatible for long term implantation and remote controller unit 108 is biocompatible for transient use both per ISO 10993. The systems are designed to have no significant interaction or interference with other electronics in any of the following modalities: implantable energy sources such as defibrillators and pacemakers; internal energy sources such as electrosurgical instruments; external energy sources such as ultrasound, x-rays and defibrillators; and radiofrequency signals such as pacemaker programmers and neurostimulators.

### EXAMPLE 1

### Implantation of a Gastric Band System

A 40 year old female is diagnosed by her clinician as obese, weighing 510 lbs. The clinician suggests to the patient that she consider a gastric banding system according to the present description. She agrees and undergoes the implantation procedure. A gastric band is implanted around her cardia thereby creating a stoma. The high precision pump unit is sutured onto the rectus muscle sheath and the tubing and reservoir passes through the rectus muscle into the peritoneal cavity and connects to the gastric band. The system comes pre-filled, so there is no need for the clinician to fill the system during the surgical procedure. The patient is sutured and sent to recovery.

### EXAMPLE 2

### Adjustment of a Gastric Band System

The female patient of Example 1, after the completion of the surgical implantation, has her gastric band system properly adjusted by her clinician. The clinician holds a remote controller unit to the skin adjacent to the rectus muscle where the high precision pump unit is located and initiates communication between the devices. An initial pressure of zero is displayed for the gastric band as no fluid has been added to the gastric band. The clinician begins to fill the gastric band using saline housed within the reservoir at a rate of about 1 cc/min and the entire filling takes less than about 5 minutes.

After filling, to about 20 psi, the patient is instructed to drink a glass of water in order to properly assess the proper inflation pressure of the gastric band to ensure it has not been over inflated. Upon confirmation that the gastric band is properly inflated, the procedure is completed and the patient returns to her normal life.

The patient instantly notices that she is much less hungry than she previously had been and is consistently consuming less food as her appetite has been decreased. She returns to her clinician's office for a follow-up visit three months after her implantation and initial gastric band filling and she has lost 20 pounds. A year later, she has lost nearly 60 lbs.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A system for facilitating obesity control comprising:
a gastric banding device including an inflatable member for containing fluid;
a fluid reservoir couplable to the inflatable member;
an implantable pump unit in communication with said fluid reservoir and effective to control pressure within said inflatable member, said implantable pump unit including a inductively powered pump and a receiver assembly in communication with the pump; and
a remote control device capable of communicating with the receiver assembly.

2. The system of claim 1 wherein the pump comprises a piezoelectric pump.

3. The system according to claim 1 wherein the pump comprises a piezoelectric diaphragm pump.

4. The system according to claim 1 wherein the pump comprises a piezoelectric membrane.

5. The system according to claim 1 wherein the pump is a unidirectional pump.

6. The system according to claim 1 wherein the implantable pump unit is passive and includes no energy storage device.

7. The system according to claim 1 wherein the implantable pump unit is entirely inductively powered.

8. The system according to claim 1 wherein the implantable pump unit further comprises a pressure sensor communicable with the remote control device.

9. The system according to claim 1 wherein the implantable pump unit further comprises an access port for enabling manual adjustment of a volume or a pressure of fluid in said inflatable member.

10. The system according to claim 9 wherein said access port is an integral part of said pump unit.

11. The system according to claim 1 wherein the implantable pump unit further comprises at least one valve in line with the pump.

12. The system according to claim 11 wherein the implantable pump unit further comprises another valve in parallel with the pump.

13. The system according to claim 12 wherein said system is MRI compatible.

14. The system according to claim 1 wherein said pump unit comprises a first fluid line including a unidirectional pump for passing fluid in a first direction and a second fluid line in parallel with said first line including a valve for passing fluid in an opposing direction.

15. An implantable high precision pump unit for use in adjusting inflation of a gastric band, the pump unit comprising:
a inductively powered, piezoelectric pump;
a first valve in line with the pump;
a second valve in parallel with the pump;
electronics in communication with the pump and first and second valves;
an override port; and
a housing containing the pump, first and second valves, electronics and override port;
the pump unit being operable without the use of an implanted energy storage device.

16. The high precision pump unit according to claim 15 controllable by an external remote control unit.

17. The high precision pump unit according to claim 15 wherein the pump is a piezoelectric diaphragm pump.

18. The high precision pump unit according to claim 15 wherein the pump is a unidirectional pump.

19. A system for facilitating obesity control comprising:
an implantable gastric banding device including an inflatable member for containing fluid and restricting a patient's cardia;
an implantable fluid reservoir;
an implantable pump unit in communication with said fluid reservoir and said gastric band device for controlling pressure within said inflatable member, said implantable pump unit including at least one piezoelectric pump and at least one valve in a parallel configuration and a receiver in direct communication with at least one piezoelectric pump and at least one valve; and
an external remote control device capable of communicating with said receiver assembly and powering said implantable pump unit.
